(19) United States
(12) Patent Application Publication
(10) Pub. No.: US 2013/0102890 A1
(43) Pub. Date: Apr. 25, 2013

(54) System and Method for Visualizing Catheter Placement in a Vasculature

(71) Applicant: Nabil Dib, Paradise Valley, AZ (US)

(72) Inventor: Nabil Dib, Paradise Valley, AZ (US)

(21) Appl. No.: 13/691,220

(22) Filed: Nov. 30, 2012

Related U.S. Application Data
(63) Continuation-in-part of application No. 12/788,194, filed on May 26, 2010.

(51) Int. Cl. A61B 5/06; A61B 8/12

(52) U.S. Cl. CPC A61B 5/061; A61B 8/12
USPC 600/424

(57) ABSTRACT

A system for advancing a needle through a vasculature to an injection site at the heart of a patient includes a guide catheter with a reflective distal tip. Also included is an imaging unit that is mounted on the catheter to radiate an energy field. Structurally, a distal portion of the catheter is biased to bend into a predetermined configuration that will position the distal end of the catheter for interception by the energy field. If necessary, coincidence of the reflective tip with the energy field is established by moving the energy field along the length of the guide catheter. With coincidence, the reflective tip reflects a signal that is useful for advancement of the needle 34b from the guide catheter and into the injection site.

This application is a continuation-in-part application of application Ser. No. 12/788,194, titled “SYSTEM AND METHOD FOR VISUALIZING CATHETER PLACEMENT IN A VASCULATURE” filed May 26, 2010 to Nabil Dib, the entire contents of which are hereby incorporated by reference herein.

### FIELD OF THE INVENTION

The present invention pertains generally to systems for advancing a needle or other secondary instrument through the vasculature of a patient to a treatment site at the heart. More particularly, the present invention pertains to systems that incorporate an imaging modality, such as ultrasound or Optical Coherence Tomography (OCT), to image a needle or other secondary instrument in the vasculature. The present invention is particularly, but not exclusively, useful as a system and method for bringing the energy field of an imaging modality into coincidence with the distal end of a catheter, to monitor the advancement of a needle, wire or other secondary instrument from the distal end of the catheter.

### BACKGROUND OF THE INVENTION

Intravascular operations are always complicated by the simple fact that there is typically no direct visual contact with the instruments that are being used to perform the operation. To help overcome this inconvenience, several effective imaging modalities have been developed for use in the vasculature. For example, ultrasound technology is a well established imaging modality that has proven useful for many applications inside a body. Optical Coherence Tomography (OCT) is another accepted imaging modality. These imaging modalities, however, have their respective unique, operational limitations that must be accounted for. In particular, the energy fields that are used by the imaging modalities must somehow be made incident on the target area that is to be imaged, and instruments to be used in the target area must be observable.

It happens that many intravascular operations can be relatively easily accomplished. Moreover, they can often be done with minimal structural manipulations. As an example, the delivery of biologics (e.g. cells, genes, protein and drugs) to a selected injection site can be easily accomplished by using a needle injector. For such an operation, however, it is essential to properly position the instrument that is being used (e.g. a needle injector). In particular, for instances wherein an imaging modality is being used to position an instrument, the energy field of the imaging modality must be positioned to both cover the injection site, and intercept (i.e. become coincident with) the instrument.

With the above in mind, it is an object of the present invention to provide a navigation system for use in advancing a needle or a wire (i.e. a guide wire) to an injection site at the heart of a patient which reconfigures a guide catheter to position its distal tip for visualization by an imaging unit. Another object of the present invention is to provide a navigation system, for use when advancing a needle or wire through the vasculature of a patient, that provides for the movement of an imaging unit so its energy field will intercept the distal tip of a guide catheter for visualization of the catheter tip at an injection site. Still another object of the present invention is to provide systems and methods for performing atrial septum procedures having the ability to image the catheter positioned in a forward looking position relative to the target tissue to reduce procedure time and increase success rate over traditional systems and methods. Yet another object of the present invention is to provide a navigation system for use in advancing a needle or wire to an injection site in the vasculature or at the heart of a patient which is simple to manufacture, is easy to use, and is cost effective.

### SUMMARY OF THE INVENTION

A system in accordance with the present invention is provided for advancing a needle to an injection site in the vasculature or at the heart of a patient. The system essentially includes a guide catheter and an imaging unit that is associated with the guide catheter. In more detail, the guide catheter has a reflective distal tip, and the imaging unit radiates an energy field in a substantially radial direction from the axis of the guide catheter for the purpose of locating the tip.

Insofar as structure of the guide catheter is concerned, a distal portion of the guide catheter is biased to bend into a predetermined configuration (i.e. the guide catheter may have a pre-bent portion). As envisioned for the present invention, this configuration will position the distal end of the catheter in the vasculature for interception by the energy field. If necessary, a coincidence of the reflective tip with the energy field can be established by manipulation of an actuator. Specifically, such a manipulation will move the energy field axially along the length of the guide catheter to intercept the reflective distal tip of the catheter. Once there is coincidence (i.e. when the reflective tip of the guide catheter is located and visualized in the energy field), the reflective tip will reflect a signal. Importantly, this reflective signal is useful for further positioning of the distal tip and for advancing the needle from the guide catheter and into the injection site. For an alternate embodiment of the present invention, the distal portion of the catheter can be steerable, rather than being pre-bent.

Structurally, the guide catheter defines an axis and it has a proximal end and a distal end. It also has a lumen that extends between the proximal and distal ends of the guide catheter. Further, the lumen is dimensioned to receive either a needle injector that includes a needle for injection into the myocardium, or a wire that passes through the lumen of the catheter to navigate the vasculature, such as by crossing heart valves or septal defects. An extracorporeal source of a fluid (e.g. biologics: cells, genes, protein and drugs) is attached to the proximal end of the injector for delivery through the needle.

An important structural aspect of the present invention is that the distal portion of the guide catheter is formed with a bendable section. Specifically, at least one part in the bendable section is biased to be bent through an angle θ. In an alternate embodiment, there can also be a second part in the bendable section that is further biased to bend through an angle φ. For the alternate embodiment, the center of rotation for the angle θ is axially opposite the center of rotation for the angle φ. Stated differently, the bendable section can be simultaneously bent in two different directions. Further, a reflective tip is attached to the bendable section at the distal end of the guide catheter, and a handle is affixed to the proximal end of the guide catheter.

Mounted on the guide catheter is an imaging unit that interacts with the reflective tip of the guide catheter to visualize the tip's location in the vasculature. In detail, the imaging unit includes a generator, a detector, and a transceiver that is mounted for axial movement on the guide catheter. Further, the imaging unit includes an actuator that is positioned in the handle of the guide catheter to move the transceiver axially along the guide catheter. The actuator will typically have a dial that is mounted on the handle, and it will include an activation wire wherein a first end of the activation wire is attached to the transceiver and a second end is engaged with the dial. Manipulation of the dial will then produce an axial movement of the transceiver along the guide catheter. Structurally, the operative components of the actuator can be selected as any one of several well-known types, such as a rack and pinion, a lead screw or a reel.

Operationally, the system of the present invention will use the generator, in combination with the transceiver, to radiate an energy field into the vasculature. This radiation will typically be in a substantially radial direction from the axis of the guide catheter. Preferably, the generator will generate ultrasound energy, but, it is well known that OCT systems can also be effective for purposes of the present invention. In either case, when the reflective tip is in the energy field, energy (e.g. ultrasound energy) will be reflected from the tip. Also, the energy will be reflected by target tissue, such as the heart. A detector that is electronically connected to the transceiver will then receive and evaluate the signal of reflected energy to determine where exactly the reflective tip is located, relative to target tissue (e.g. heart), in the energy field. The needle injector can then be advanced through the lumen of the guide catheter for extension of the needle beyond the reflective tip and from the distal end of the guide catheter for use at an injection site. As indicated above, a guide wire, rather than the needle injector, may be advanced through the catheter.

In another aspect, a system for performing a procedure on targeted heart tissue of a patient with a secondary instrument is described that includes a catheter formed with a lumen that has a pre-bent or actively bendable section that is located along a distal portion of the catheter. The secondary instrument can be inserted into the lumen of the catheter for advancement therein to extend at least a portion of the secondary instrument beyond a distal end of the catheter. For example, the secondary instrument can be a needle injector, electrophysiology ablation catheter or a delivery catheter for delivering an embolic protection device or some other device. Also, an imaging unit transceiver is coupled with the catheter to radiate an energy field in a substantially radial direction from the axis. With this arrangement, the imaging unit is able to simultaneously image a reflective tip on the distal end of the catheter, the secondary instrument and the targeted heart tissue.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic drawing of a system in accordance with the present invention;

FIG. 2 is a cross sectional view of the distal portion of the guide catheter of the present invention as seen along the line 2-2 in FIG. 1;

FIG. 3A is a view of the distal portion of the guide catheter shown in its operational environment and configured with a single bend used for positioning the catheter's distal end at an injection site;

FIG. 3B is a view of the distal portion of the guide catheter shown in its operational environment and configured with a double bend used for positioning the catheter's distal end at an injection site;

FIG. 4A shows a rack and pinion arrangement for the actuator of the present invention;

FIG. 4B shows a lead screw arrangement for the actuator of the present invention;

FIG. 4C shows a reel arrangement for the actuator of the present invention;

FIG. 5 is a view of the distal portion of another embodiment of a catheter, shown in its operational environment and configured with a double bend for positioning the catheter's distal end at a treatment site;

FIG. 6 is a view of the distal portion of another embodiment of a catheter, shown in its operational environment and configured with a single bend and an extended ultrasound transceiver for positioning the catheter's distal end at a treatment site;

FIG. 7 is a view of the distal portion of another embodiment of a catheter, shown in its operational environment and configured with a double bend and an extended ultrasound transceiver for positioning the catheter's distal end at a treatment site;

FIG. 8 is a sectional view as in FIG. 2 showing an injector having a needle, dilator and needle sheath;

FIG. 9 is a perspective view of an injection needle having a spiral pattern laser cut on its exterior surface to increase flexibility and/or ultrasound reflectivity;

FIG. 10 is a view of the distal portion of another embodiment of a catheter, shown in its operational environment and configured with a pigtail bend for positioning the catheter's distal end at a treatment site and within the observable energy field of an ultrasound transceiver; and

FIG. 11 is a view of the distal portion of another embodiment of a catheter, shown in its operational environment and configured with a bend having a substantially straight portion between two curved portions for positioning the catheter's distal end at a treatment site and within the observable energy field of an ultrasound transceiver.

### DESCRIPTION OF THE PREFERRED EMBODIMENTS

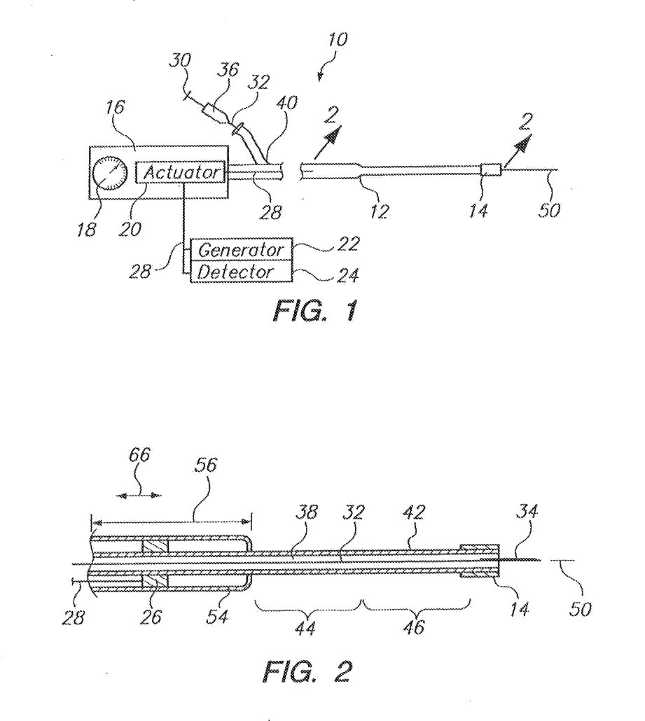

Referring initially to FIG. 1, a system in accordance with the present invention is shown and is generally designated 10. As shown, the system 10 includes a guide catheter 12 that has a reflective tip 14 at its distal end. The system 10 also has a handle 16 that is mounted at the proximal end of the guide catheter 12, with a dial 18 and an actuator 20 being included as part of the handle 16. Structurally, the dial 18 is connected directly to the actuator 20 for manipulating the actuator 20 during an operation of the system 10. FIG. 1 further indicates that the system 10 includes an energy generator 22 and a detector 24. More specifically, with cross reference to FIG. 2, it is to be appreciated that both the energy generator 22 and the detector 24 are electronically connected to a transceiver 26 via an activation wire 28. It is also to be appreciated that the activation wire 28 can be manipulated by the actuator 20 to move the transceiver 26. Collectively, the energy generator 22, detector 24 and the transceiver 26 are hereinafter sometimes referred to as an imaging unit.

Still referring to FIG. 1, it will be seen that the guide catheter 12 is to be used with a needle injector 30. More specifically, the needle injector 30 includes a needle wire 32 that has a needle 34 formed at its distal end (see FIG. 2). A fluid source 36 is also provided for the injector 30, and this source 36 will typically hold a fluid that includes biologics (e.g. cells, genes, protein and drugs) for delivery through the injector 30. As shown, access into the lumen 38 (see FIG. 2) of the guide catheter 12 for both the needle 34 and the needle wire 32 of the injector 30 is provided via a y-site 40.

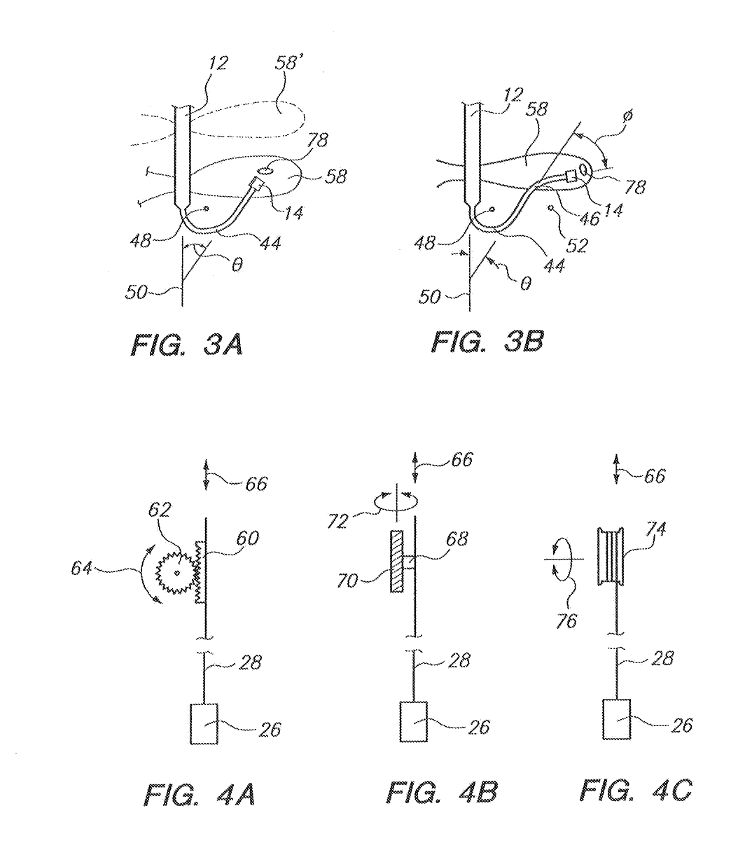

An important structural aspect of the guide catheter 12 is its ability to be reconfigured. This will be best appreciated with reference to FIG. 2, along with reference to FIGS. 3A and 3B. In FIG. 2 it is shown that a bendable section 42, at the distal portion of the guide catheter 12, can be considered as having at least one reconfigurable part 44. Alternatively, there can be an additional reconfigurable part 46. Consider first, a structure for the guide catheter 12 wherein there is no part 46 and, instead, only a part 44. With reference to FIG. 3A, it will be seen that for this embodiment of the guide catheter 12, the bendable section 42 can be biased to bend around a center of curvature 48 to establish an angle θ. As shown, the angle θ is measured relative to an axis 50 that is generally defined by the length of the guide catheter 12. On the other hand, as shown in FIG. 3B, when parts 44 and 46 are both incorporated into the bendable section 42 of the guide catheter 12, the bendable section 42 can be respectively biased to rotate through the angle θ and, additionally, through an angle φ around a center of curvature 52. As shown, the angles θ and φ are measured in opposite directions with their respective centers of curvature 48 and 52 on opposite sides of the axis 50. In addition to providing for structural biasing in the bendable section 42, it is well known that various devices have been proposed for bending or steering a catheter through the vasculature of a patient (not shown). For purposes of the present invention, any such device would be suitable for reconfiguring the guide catheter 12.

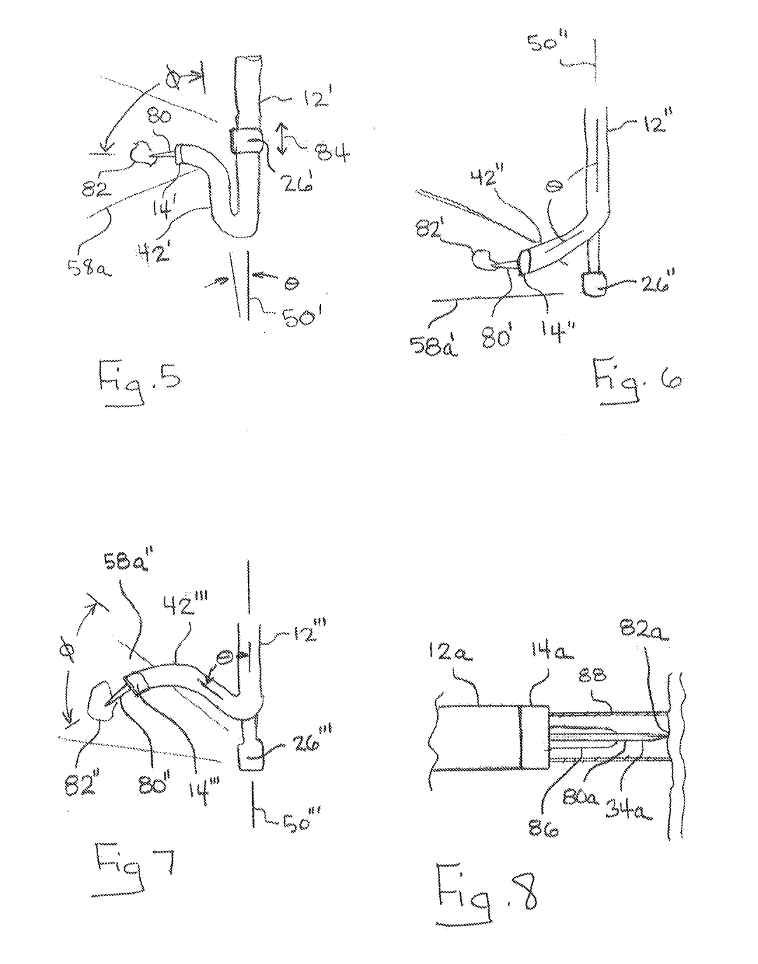

Another structural aspect of the guide catheter 12 that is of more general importance for the entirety of the system 10 concerns the actuator 20. More specifically, the manipulation of the imaging unit and the consequent movement of the transceiver 26 is essential for the operation of the system 10. This aspect will be best appreciated by sequentially cross referencing FIG. 2 with FIGS. 4A, 4B and 4C. Specifically, this aspect regards movements of the transceiver 26 along the axis 50 of the guide catheter 12.

With reference to FIG. 2 it will be noted that, proximal to its bendable section 42, the guide catheter 12 is formed with a sleeve 54. Further, it is to be understood that the transceiver 26 is moveable inside the sleeve 54 by a manipulation of the actuator 20. More specifically, movements of the transceiver 26 by the actuator 20 are made on the guide catheter 12, through a range 56, in directions back and forth along the axis 50 indicated by arrows 66. The real purpose here is to move an energy field 58 (i.e. transceiver 26) that is radiated from the transceiver 26. In detail, the energy field 58 will be primarily oriented in a direction perpendicular to the axis 50, and will be radiated whenever the transceiver 26 is activated by the generator 22. As envisioned for the present invention, although the generator 22 will preferably generate ultrasound energy, any other type of energy field that is known for use as an imaging modality is suitable (e.g. OCT). Further, although a two-dimensional field of ultrasound energy is typical, a three-dimensional ultrasound field may also be used.

In accordance with the system 10, several different types of mechanisms can be incorporated into the actuator 20 for the purpose of moving the energy field 58. The mechanisms shown in FIGS. 4A, 4B and 4C are only exemplary. In FIG. 4A, the components for the actuator 20 are shown to include a straight toothed rack 60 that is affixed to the activation wire 28. A pinion 62 is shown engaged with the rack 60 and, with this engagement, the pinion 62 can be rotated by the dial 18 on handle 16 in the directions indicated by arrows 64. This rotation of the pinion 62 will then move the transceiver 26 axially along the guide catheter 12 in the directions of arrows 66. In another arrangement of components for the actuator 20 shown in FIG. 4B, a projection 68 is affixed to the activation wire 28. A lead screw 70 is then engaged with the projection 68. Consequently, a rotation of the lead screw 70 by the dial 18 in directions indicated by arrows 72 will move the transceiver 26 axially along the guide catheter 12 in the directions of arrows 66. Further, in another embodiment of components for the actuator 20 shown in FIG. 4C, a reel 74, is incorporated to take-up the activation wire 28. More specifically, with a rotation of the reel 74 in the directions indicated by arrows 76, the transceiver 26 will move axially along the guide catheter 12 in the directions of arrows 66.

For an operation of the system 10, the guide catheter 12 is positioned in the vasculature of a patient (not shown), and there it is reconfigured as shown in either FIG. 3A or FIG. 3B. The transceiver 26 can then be moved by the actuator 20, as disclosed above, so that the energy field 58 radiated by the transceiver 26 will intercept the reflective tip 14 of the guide catheter 12. For example, such a movement of the energy field 58 is shown in FIG. 3A where it can be seen that the energy field 58′ has been moved axially along the guide catheter 12 to a new position for the energy field 58. Once there is coincidence (i.e. the reflective tip 14 of the guide catheter 12 is located in the energy field 58, and can be visualized with the detector 24 of the particular imaging modality being used), the reflective tip 14 can be further manipulated. Also, in this configuration the reflective tip 14 is positioned so that an advancement of needle 34 (or a guide wire) from reflective tip 14 will be seen as an axial movement of the needle 34. Further, because the energy field 58 will also see an injection site 78 on target tissue (e.g. the heart), advancement of the needle 34 can be made relative to the injection site 78 (target tissue). In particular, this additional manipulation may be necessary in order to properly position the reflective tip 14 at a predetermined injection site 78. The needle injector 30 can then be advanced through the guide catheter 12 to perform an injection with the needle 34 at the injection site 78.

FIG. 5 shows the distal end of another embodiment of a catheter 12′ having a reflective tip 14′ and bendable section 42′. As shown, for this embodiment, the bendable section 42′ can be configured as a compound curve at the distal portion of the catheter 12′. FIG. 5 further shows that a secondary instrument 80, such as the needle injector described above or some other type of secondary instrument (see below), can be extended from the lumen of the catheter 12′ and beyond the distal end of the catheter 12′ for interaction with target tissue 82. Depending on the type of procedure, the secondary instrument 80 can be an injection catheter as described above, a needle and or needle/dilator assembly (see FIG. 8) for example to puncture and or cross the atrial septum, a stylet, an electrophysiology ablation catheter or some other type of ablation catheter known in the pertinent art to ablate target tissue, a delivery catheter for delivering an embolic protection device, a snare or some other device or implant, a guidewire, for example, for crossing a heart valve, atrial septum or ventricular septum or any other secondary instrument known in the pertinent art.

Continuing with FIG. 5, it can be seen that the catheter 12′ includes a transceiver 26′, as described above, that is positioned on the catheter 12′ proximal to the bendable section 42′, for producing an energy field 58a. For example, the transceiver 26′ can be a phased array transceiver having a plurality of individually controllable ultrasound transducers. With this arrangement, the shape, and in some cases the direction of the ultrasound energy field 58a emitted by the transceiver 26′ can be controlled by activating the phased array transceiver 26′ with the appropriate drive signal(s). As shown, the transceiver 26′ can be configured to produce a substantially cone shaped energy field 58a. It is to be appreciated that within the cone, suitable imaging may be performed. Typically, the ultrasound energy is able to look through and image behind standard catheter materials including braided materials. Also shown in FIG. 5, the cone shaped energy field 58a can extend in a substantially radial direction relative to the catheter axis 50′. In some implementations of the catheter 12′, the transceiver 26′ can be moveable, as described above, back and forth along the axis 50′ (see arrow 84) to selectively move the energy field 58a and intercept the reflective tip 14′, secondary instrument 80 and target tissue 82 in a single image. In addition, in some implementations, the transceiver 26′ can be rotated about the axis 50′ to selectively move the energy field 58a to a desired location. Alternatively, a transceiver 26′ producing another type of energy field known in the pertinent art for use as an imaging modality, such as OCT, may be used.

For the catheter 12′ shown in FIG. 5, the bendable section 42′ can be biased to establish an angle, θ, measured relative to an axis 50′ (i.e. the axis 50′ is generally defined by the straight portion of the catheter 12′ proximal to the bendable section 42′), and, additionally, biased to establish an angle φ (as described above with reference to FIG. 3B). For the embodiment shown in FIG. 5, the angle, θ is typically in the range of 0 degrees <θ≦90 degrees, and the angle, φ is typically in the range of 0 degrees <φ≦90 degrees to place the reflective tip 14′, secondary instrument 80 and target tissue 82 in the observable portion of the energy field 58a, as shown. More typically, as shown, an angle, θ greater than about 160 degrees and an angle, φ greater than about 70 degrees is used for the embodiment shown in FIG. 5. The compound bend can be a single plane curve, as shown, or, a bi-plane curve may be used. With the arrangement shown, an image can be produced using the transceiver 26′ in which the secondary instrument 80 is positioned in a forward looking position relative to the target tissue 82.

For the embodiment shown in FIG. 5, the compound curve can be established using a pre-bent section 42′. In this case, the pre-bent section 42′ can be delivered over a guidewire which straightens the section for navigation through the vasculature. At the location of the procedure, the section 42′ will assume its pre-bent shape as the guidewire is retracted. Alternatively, the section 42′ can be actively deflected at the treatment site. For example, to create the compound curve the catheter 12′ can include a pair of wires (not shown) extending along the length of the catheter 12′ and connected to respective anchor rings (not shown) located at the distal end of each curve making up the compound curve.

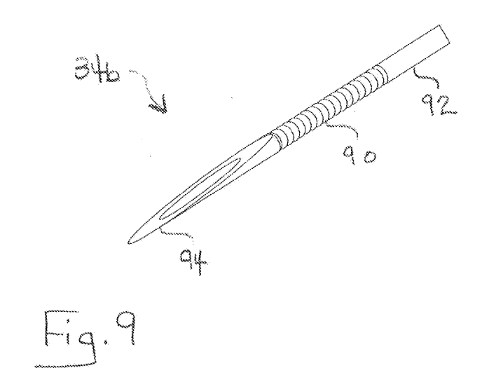

FIG. 6 shows the distal end of another embodiment of a catheter 12″ having a reflective tip 14″ and bendable section 42″ that can be placed in a so-called “hockey stick” shape. As shown, for this embodiment, the bendable section 42″ can be configured as a single curve at the distal portion of the catheter 12″. FIG. 6 further shows that a secondary instrument 80′, such as the needle injector described above or some other type of secondary instrument (described above), can be extended from the lumen of the catheter 12″ and beyond the distal end of the catheter 12″ for interaction with target tissue 82′.

Continuing with FIG. 6, it can be seen that the catheter 12″ includes a transceiver 26″, as described above, for producing an energy field 58a′ which, as shown, can be a substantially fan or coned shaped energy field 58a′. It is to be appreciated that within the cone, suitable imaging may be performed allowing for dynamic monitoring of the catheter 12″, reflective tip 14″ and target tissue 82′. Also shown in FIG. 6, the cone shaped energy field 58a′ can extend in a substantially radial direction relative to the catheter axis 50″. FIG. 6 shows that the transceiver 26″ is integral with the catheter 12″. For example, the transceiver 26″ can extend from a lumen of the catheter 12″, or both the catheter 12″ and transceiver 26″ can be delivered to the treatment site in a common guide catheter (not shown). For both cases, as shown, the transceiver 26″ can be extended from a location proximal to the bendable section 42″ and along axis 50″ to a location spaced from the catheter 12″. In some implementations the transceiver 26″ can be moveable, as described above, back and forth along the axis 50″ to selectively move the energy field 58a′ and intercept the reflective tip 14″, secondary instrument 80′ and target tissue 82′ in a single image. In addition, in some implementations, the transceiver 26″ can be rotated about the axis 50″ to selectively move the energy field 58a′ to a desired location. Alternatively, a transceiver 26″ producing another type of energy field known in the pertinent art for use as an imaging modality, such as OCT, may be used.

For the catheter 12″ shown in FIG. 6, the bendable section 42″ can be biased to establish an angle, θ, (as described above with reference to FIG. 3A) measured relative to an axis 50″ (i.e. the axis 50″ is generally defined by the straight portion of the catheter 12″ proximal to the bendable section 42″). For the embodiment shown in FIG. 6, the angle, θ is typically in the range of 90 degrees <θ<180 degrees to distance the reflective tip 14″ from the transceiver 26″ and place the reflective tip 14″, secondary instrument 80′ and target tissue 82′ in the observable portion of the energy field 58a′, as shown.

More typically, as shown, an angle in the range of 125 degrees <θ<145 degrees is used for the embodiment shown in FIG. 6. With the arrangement shown, an image can be produced using the transceiver 26″ in which the secondary instrument 80′ is positioned in a forward looking position relative to the target tissue 82′. For the embodiment shown in FIG. 6, the curve can be established using a pre-bent bendable section 42″ or can be actively deflected at the treatment site.

FIG. 7 shows the distal end of another embodiment of a catheter 12′″ having a reflective tip 14′″ and bendable section 42′″. As shown, for this embodiment, the bendable section 42′″ can be configured as a compound curve at the distal portion of the catheter 12′″. FIG. 7 further shows that a secondary instrument 80″, such as the needle injector (described above) or some other type of secondary instrument (described above), can be extended from the lumen of the catheter 12′″ and beyond the distal end of the catheter 12′″ for interaction with target tissue 82″.

Continuing with FIG. 7, it can be seen that the catheter 12′″ includes a transceiver 26′″, as described above, for producing an energy field 58a″ which, as shown, can be a substantially coned shaped energy field 58a″. It is to be appreciated that within the cone, suitable imaging may be performed. Also shown in FIG. 7, the cone shaped energy field 58a″ can extend in a substantially radial direction relative to the catheter axis 50′″. FIG. 7 shows that the transceiver 26′″ is integral with the catheter 12′″. For example, the transceiver 26′″ can extend from a lumen of the catheter 12′″ or both the catheter 12′″ can both be delivered to the treatment site in a common guide catheter (not shown). For both cases, as shown, the transceiver 26′″ can be extended from a location proximal to the bendable section 42′″ and along axis 50′″ to a location spaced from the catheter 12′″. In some implementations the transceiver 26′″ can be moveable, as described above, back and forth along the axis 50′″ to selectively move the energy field 58a″ and intercept the reflective tip 14′″, secondary instrument 80″ and target tissue 82″ in a single image. In addition, in some implementations, the transceiver 26′ can be rotated about the axis 50′″ to selectively move the energy field 58a″ to a desired location. Alternatively, a transceiver 26′″ producing another type of energy field known in the pertinent art for use as an imaging modality, such as OCT, may be used.

For the catheter 12′″ shown in FIG. 7, the bendable section 42′″ can be biased to establish an angle, θ, measured relative to an axis 50′″ (i.e. the axis 50′″ is generally defined by the straight portion of the catheter 12′″ proximal to the bendable section 42′″), and, additionally, biased to establish an angle φ (as described above with reference to FIG. 31). For the embodiment shown in FIG. 7, the angle, θ is typically in the range of 0 degrees <θ≦90 degrees, and the angle, φ is typically in the range of 0 degrees <φ≦90 degrees to place the reflective tip 14′″, secondary instrument 80″ and target tissue 82″ in the observable portion of the energy field 58a″, as shown. More typically, as shown, an angle, θ in the range of 35 degrees <θ≦55 degrees and an angle, φ in the range of 30 degrees <φ≦60 degrees is used for the embodiment shown in FIG. 7. The compound bend can be a single plane curve, as shown, or, a bi-plane curve may be used. With the arrangement shown, an image can be produced using the transceiver 26′″ in which the secondary instrument 80″ is positioned in a forward looking position relative to the target tissue 82″. For the embodiment shown in FIG. 7, the compound curve can be established using a pre-bent bendable section 42″ or can be actively deflected at the treatment site.

FIG. 8 shows an embodiment of a secondary instrument 80a for use in any of the embodiments described above (i.e. FIGS. 3A, 3B and 5-7) to pierce and dilate target tissue 82a such as an atrial or ventricular septum. As shown in FIG. 8, the secondary instrument 80a includes a needle 34a that extends distally from the reflective tip 14a of catheter 12a. Secondary instrument 80a also includes a dilator 86 and sheath 88. For the embodiments described herein, the needle, including injection needles and puncturing needles, can include a needle tip that is straight, curved (not shown) or can include a one or more pre-defined bends (not shown). These curves and/or bends are typically on the section of the needle that will extend distally from the distal end of the catheter. Continuing with FIG. 8, the sheath 88, for example, may be shaped as a tube and made of a polymer, a reinforced polymer or metal. In use, the distal end of the catheter 12a is positioned relative to the target tissue. With the catheter 12a positioned, the sheath 88 can be advanced distally, telescoping out from the distal end of catheter 12a until the sheath contacts the target tissue 82a. Next, the needle 34a can be advanced distally to puncture the target tissue 82a, (e.g. atrial suptum) while the sheath 88 protects the needle 34a against damage to the needle 34a or collateral (i.e. non-target) tissue. Once the target tissue 82a is punctured, the dilator 86 can be distally advanced through the hole made by the needle 34a to dilate the hole to a desired size. It is to be appreciated that the sheath 88 described herein can also be used with the injector systems (described above) to deliver a medicament or cell therapy to target tissue. The integration of the transceiver 26″ with the needle 34a allows the user to see the depth of the needle 34a in the tissue 82a in relation to anatomical landmarks and boundaries, and allows the user to see the interaction of the needle sheath 88 and needle 34a with the anatomic wall of the heart or other anatomical structure. This helps the user watch the impact of the pressure against the wall (i.e. condensing/displacing tissue) and helps reduce the risk of puncturing through a wall. It also helps the user see penetration and depth of needle 34a to insure injection of stem cells/biologic material to the right target area. In some cases, the needle may not be hollow all the way through, but may have a solid core until the tip, allowing for more pushability and control. In addition, for some embodiments the needle and/or needle sheath may have some steering mechanism and also may be preshaped and deflectable to help align the needle in the field of view of the imaging window. In an alternative embodiment, the injector can include a plurality of needles coming out as a bundle for infusion.

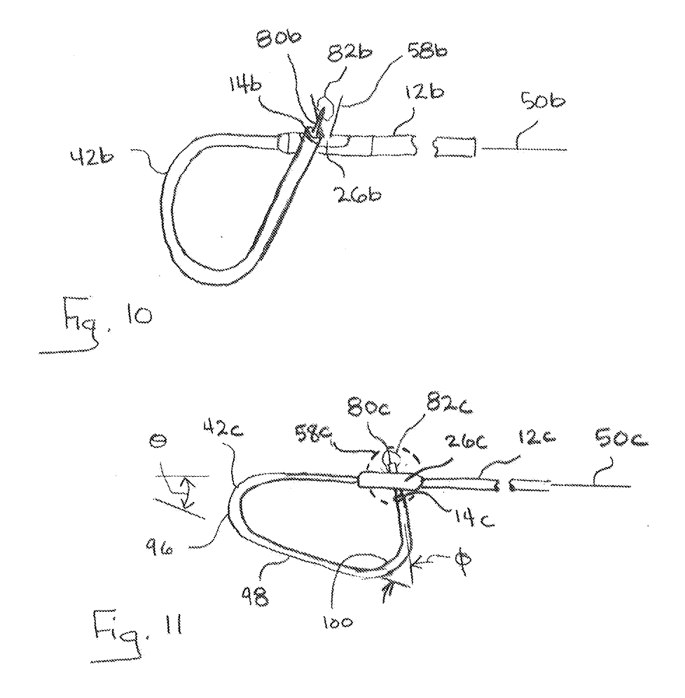

FIG. 9 shows an injection needle 34b that has been treated to increase the needle 34b's reflection of ultrasonic energy, and thus, increase the observability of the needle 34b when used with the imaging units described herein. Untreated, the relatively thin needles used for treating heart tissues, having a thickness of about AWG 27-28 and made of stainless steel or nitanol, are often difficult to observe using ultrasound. Preferably, the needle 34b is observable when advancing, penetrating and injecting inside the tissue/organ/muscle. As shown in FIG. 9, a pattern 90 can be scribed on the exterior surface 92 of needle 34b to increase the surface area and increase the amount of needle surface area that reflects energy back, along a particular angle, to a transceiver (not shown). For example, the pattern 90 may be scribed onto the surface using a laser, a suitable machining process or cut with another similar process. As shown, the pattern 90 may consist of a spiral that extends along a portion of the needle 34b, near the sharp distal tip 94. Other suitable patterns can include one or more spaced apart rings formed on the surface 92 (not shown). In addition, surface features may be established on the surface 92 of the needle 34b to increase the flexibility of the needle 34b, allowing the needle 34b to navigate through the vasculature. Alternatively, or in addition to the scribed pattern 90, a coating can be applied to a portion or all of the surface 92 of the needle 34b (not shown) to increase ultrasound reflectivity and observability. For example, an echogenic polymer coating manufactured by (STS Biopolymers, Henrietta, N.Y.) which produces a polymer film having a porous microstructure that entraps microbubbles of air may be used to increase needle observability.

FIG. 10 shows the distal end of another embodiment of a catheter 12b having a reflective tip 14b and bendable section 42b. As shown, for this embodiment, the bendable section 42b can be configured as a so-called “pigtail curve” creating a full loop at the distal portion of the catheter 12b. FIG. 10 further shows that a secondary instrument 80b, such as the needle injector (described above) or some other type of secondary instrument (described above), can be extended from the lumen of the catheter 12b and beyond the distal end of the catheter 12b for interaction with target tissue 82b.

Continuing with FIG. 10, it can be seen that the catheter 12b includes a transceiver 26b, as described above, for producing an energy field 58b which, as shown, can be a substantially coned shaped energy field 58b. It is to be appreciated that within the cone, suitable imaging may be performed. Also shown in FIG. 10, the cone shaped energy field 58b can extend in a substantially radial direction relative to the catheter axis 50b. FIG. 10 shows that the transceiver 26b is mounted on the catheter 12b proximal to the bendable section 42b and is thus integral with the catheter 12b. In some implementations the transceiver 26b can be moveable, as described above, back and forth along the axis 50b to selectively move the energy field 58b and intercept the reflective tip 14b, secondary instrument 80b and target tissue 82b in a single image. In addition, in some implementations, the transceiver 26b can be rotated about the axis 50b to selectively move the energy field 58b to a desired location. Alternatively, a transceiver 26b producing another type of energy field known in the pertinent art for use as an imaging modality, such as OCT, may be used.

For the catheter 12b shown in FIG. 10, the bendable section 42b is biased to bend greater than 180 degrees such that the distal end of the catheter 12b approaches or crosses a portion of the catheter 12b proximal to the bendable section 42b to position the reflective tip 14b, secondary instrument 80b and target tissue 82b in the observable portion of the energy field 58b, as shown. For the embodiment shown in FIG. 10, the pigtail curve can be established using a pre-bent bendable section 42b or can be actively deflected at the treatment site.

FIG. 10 shows the distal end of another embodiment of a catheter 12b having a reflective tip 14b and bendable section 42b. As shown, for this embodiment, the bendable section 42b can be configured as a so-called “pigtail curve” creating a full loop at the distal portion of the catheter 12. FIG. 10 further shows that a secondary instrument 80b, such as the needle injector (described above) or some other type of secondary instrument (described above), can be extended from the lumen of the catheter 12b and beyond the distal end of the catheter 12b for interaction with target tissue 82b.

Continuing with FIG. 10, it can be seen that the catheter 12b includes a transceiver 26b, as described above, for producing an energy field 58b which, as shown, can be a substantially coned shaped energy field 58b. It is to be appreciated that within the cone, suitable imaging may be performed. Also shown in FIG. 10, the cone shaped energy field 58b can extend in a substantially radial direction relative to the catheter axis 50b. FIG. 10 shows that the transceiver 26b is mounted on the catheter 12b proximal to the bendable section 42b and is thus integral with the catheter 12b. In some implementations the transceiver 26b can be moveable, as described above, back and forth along the axis 50b to selectively move the energy field 58b and intercept the reflective tip 14b, secondary instrument 80b and target tissue 82b in a single image. In addition, in some implementations, the transceiver 26b can be rotated about the axis 50b to selectively move the energy field 58b to a desired location. Alternatively, a transceiver 26b producing another type of energy field known in the pertinent art for use as an imaging modality, such as OCT, may be used.

For the catheter 12b shown in FIG. 10, the bendable section 42b is biased to bend greater than 180 degrees such that the distal end of the catheter 12b approaches or crosses a portion of the catheter 12b proximal to the bendable section 42b to position the reflective tip 14b, secondary instrument 80b and target tissue 82b in the observable portion of the energy field 58b, as shown. For the embodiment shown in FIG. 10, the pigtail curve can be established using a pre-bent bendable section 42b or can be actively deflected at the treatment site.

FIG. 11 shows the distal end of another embodiment of a catheter 12c having a reflective tip 14c and bendable section 42c. As shown, for this embodiment, the bendable section 42c can create a full loop at the distal portion of the catheter 12c. FIG. 11 further shows that a secondary instrument 80c, such as the needle injector (described above) or some other type of secondary instrument (described above), can be extended from the lumen of the catheter 12c and beyond the distal end of the catheter 12c for interaction with target tissue 82c.

Continuing with FIG. 11, it can be seen that the catheter 12c includes a transceiver 26c, as described above, for producing an energy field 58c which, as shown, can be a substantially coned shaped energy field 58c (field is oriented into the plane of the page). It is to be appreciated that within the cone, suitable imaging may be performed. Also shown in FIG. 11, the cone shaped energy field 58c can extend in a substantially radial direction relative to the catheter axis 50c. FIG. 11 shows that the transceiver 26c is mounted on the catheter 12c proximal to the bendable section 42c and is thus integral with the catheter 12c. In some implementations the transceiver 26c can be moveable, as described above, back and forth along the axis 50c to selectively move the energy field 58c and intercept the reflective tip 14c, secondary instrument 80c and target tissue 82c in a single image. In addition, in some implementations, the transceiver 26c can be rotated about the axis 50c to selectively move the energy field 58c to a desired location. Alternatively, a transceiver 26c producing another type of energy field known in the pertinent art for use as an imaging modality, such as OCT, may be used.

For the catheter 12c shown in FIG. 11, the bendable section 42c includes portions 96, 98 and 100. As shown, portions 96 and 100 are curved portions and portion 98 is substantially straight. As further shown, portion 96 can be biased to establish an angle, θ, measured relative to an axis 50c (i.e. the axis 50c is generally defined by the straight portion of the catheter 12c proximal to the bendable section 42c), and, additionally, portion 100 can be biased to establish an angle φ (as described above with reference to FIG. 3B). For the embodiment shown in FIG. 11, the angle, θ is typically in the range of 0 degrees <θ≦90 degrees, and the angle, φ is typically in the range of 0 degrees <φ≦180 degrees to place the reflective tip 14c, secondary instrument 80c and target tissue 82c in the observable portion of the energy field 58c, as shown. More typically, as shown, an angle, θ in the range of 35 degrees <θ≦55 degrees and an angle, φ in the range of 45 degrees <θ≦90 degrees is used for the embodiment shown in FIG. 10, The compound bend can be a single plane curve with a small out of plane curve to allow the secondary instrument 80c to cross the catheter 12c, as shown, or, a more pronounced bi-plane curve may be used. For the embodiment shown in FIG. 11, the curve can be established using a pre-bent bendable section 42c or can be actively deflected at the treatment site.

Applications of the systems described above include procedures/treatments of the atrial septum. These treatments include puncturing the atrial septum, crossing the atrial septum with a wire (for example, to perform mitral valve repair or atrial ablation), atrial septal defect (ASD) closure and patent foramen ovale (PFO) closure. In the past, these atrial septum procedures have had extremely low success rates, a lengthy procedure time (e.g. 10-30 minutes) and have often resulted in undesirable perforations of the septum. These shortcomings have been attributed to poor imaging of the catheter and secondary instruments using a nonintegrated imaging system (i.e. a system in which the ultrasound transceiver is not integrated with the catheter/secondary instrument). Using the imaging system as described herein, and in particular, the ability to image the catheter tip, secondary instrument and target tissue in a single image, with the catheter positioned in a forward looking position relative to the target tissue can reduce procedure time and increase success rate.

Additional applications of the systems described above include crossing the aortic valve, delivering cells, such as stem cells, or other medicaments to the endocardium (see description above), crossing the ventricular septum and repairing or replacing a heart valve.

The systems described herein are compatible with other imaging systems found in a modern cathlab and can be used together with one or more of these other imaging systems to accurately deliver and view the needle or other secondary instrument as it is introduced into the heart or other tissue. These other imaging systems include, but are not limited to, 2D ultrasound, 3D ultrasound, MRI, MRI integrated picture, the NOGA mapping system (Cordis), angiography, CT, PET/nuclear imaging, a 3D mapping system, 3D left ventricle angiogram and 3D echocardiogram.

While the particular System and Method for Visualizing Catheter Placement in a Vasculature as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for performing a procedure on targeted heart tissue of a patient with a secondary instrument, the system comprising:
a catheter defining an axis and having a proximal end and a distal end, wherein the catheter is formed with a lumen extending between the ends thereof for extending at least a portion of the secondary instrument beyond the distal end of the catheter, and wherein the catheter has a bendable section located along a distal portion of the catheter;
a reflective tip attached to the bendable section at the distal end of the catheter; and
an imaging unit transceiver coupled with the catheter to radiate an energy field in a substantially radial direction from the axis of the catheter, the imaging unit transceiver for simultaneously imaging the reflective tip, the secondary instrument and the targeted heart tissue.

2. A system as recited in claim 1 wherein the secondary instrument is a needle injector having a reflective needle.

3. A system as recited in claim 1 wherein the reflective needle has an exterior surface and includes surface features on the exterior surface to increase the reflectivity of the needle.

4. A system as recited in claim 1 wherein the needle injector further comprises a needle sheath advanceable beyond the distal end of the catheter.

5. A system as recited in claim 1 wherein the secondary instrument is an electrophysiology ablation catheter.

6. A system as recited in claim 1 wherein the secondary instrument is a delivery catheter for delivering an embolic protection device.

7. A system as recited in claim 1 wherein the secondary instrument comprises a needle and a dilator.

8. A system as recited in claim 1 further wherein the bendable section of the guide catheter is pre-bent.

9. A system as recited in claim 1 further comprising an actuator for moving the imaging unit transceiver to intercept the reflective tip with the energy field to create a signal from the reflective tip for receipt by the imaging unit transceiver to determine where the reflective tip is located in the energy field.

10. A system as recited in claim 1 wherein the imaging unit transceiver comprises a phased array transceiver having a plurality of transducers.

11. A system as recited in claim 1 further comprising:
an imaging unit generator electronically connected to the transceiver for generating energy for the energy field; and
an imaging unit detector electronically connected to the imaging unit transceiver for receiving and evaluating reflected energy signal.

12. A system as recited in claim 11 wherein the imaging unit generator generates ultrasound energy for the energy field.

13. A system as recited in claim 11 wherein the imaging unit generator generates Optical Coherence Tomography (OCT) energy for the energy field.

14. A system as recited in claim 1 wherein the bendable section of the catheter is biased to be bent around a center of rotation through an angle θ to position the reflective tip in the energy field.

15. A system as recited in claim 14 wherein the center of rotation for the angle θ is a first center of rotation, and a first part of the bendable section is bent through the angle θ, and wherein a second part of the bendable section is further biased to bend around a second center of rotation through an angle φ, and further wherein the first center of rotation is axially opposite the second center of rotation.

16. A system for performing a procedure on targeted heart tissue of a patient which comprises:
a catheter means defining an axis and having a proximal end and a distal end, wherein the catheter means is formed with a lumen extending between the ends thereof, and wherein the catheter means has a bendable section located along a distal portion of the catheter means;
an instrument means insertable into the lumen of the catheter means for advancement therein to extend a reflective portion of the instrument means beyond the distal end of the catheter means;
a means for reflecting energy attached to the bendable section at the distal end of the catheter means; and
a transceiver means for use as part of an imaging unit, the transceiver means mounted on the catheter means to radiate an energy field in a substantially radial direction from the axis of the catheter means, the transceiver means for simultaneously imaging the means for reflecting energy, the reflective portion of the instrument means and the target tissue.

17. A method for performing a procedure on targeted heart tissue of a patient which comprises the steps of:
positioning a distal end of a catheter in a patient,
activating and imaging unit transceiver integrally coupled with the catheter to radiate an energy field in a substantially radial direction from the axis of the catheter;
bending a distal portion of the catheter to place a reflective tip attached to the bendable section of the guide catheter at a position to reflect the energy field;
advancing a secondary instrument through a lumen of the catheter to extend a reflective portion of the secondary instrument beyond the distal end of the catheter; and
simultaneously imaging the reflective tip, the reflective portion of the secondary instrument and the target tissue.

18. A method as recited in claim 17 wherein the procedure is a cell therapy injection procedure.

19. A method as recited in claim 17 wherein the procedure is a tissue ablation procedure.

20. A method as recited in claim 17 wherein the procedure is an atrial septal crossing procedure.
